(19) United States
(12) Patent Application Publication
(10) Pub. No.: US 2018/0119083 A1
(43) Pub. Date: May 3, 2018

(54) Airlift Reactor Assembly with Helical Sieve Plate

(71) Applicant: Jiangnan University, Wuxi (CN)

(72) Inventors: Zhiyong Zheng, Wuxi (CN); Yuqi Chen, Wuxi (CN); Xiaobei Zhan, Wuxi (CN); Minjie Gao, Wuxi (CN)

(21) Appl. No.: 15/795,320

(22) Filed: Oct. 27, 2017

(30) Foreign Application Priority Data
Oct. 28, 2016 (CN) 201610956295.9

(51) Int. Cl. C12M 1/00; B01J 8/08; B01J 19/24; B01J 10/00; C12M 1/08

(52) U.S. Cl. CPC C12M 29/08; B01J 8/08; B01J 19/24; B01J 10/002; B01J 2219/24; C12M 27/20; C12M 27/24; B01J 2208/00884; C12M 23/22

(57) ABSTRACT

The present invention discloses an airlift reactor assembly with a helical sieve plate, comprising a reaction tank, wherein a draft tube and a gas sparger are assembled in the reaction tank, the gas sparger is arranged just below an riser section of the draft tube, a helical sieve plate is arranged in the riser section of the draft tube, and a body of the helical sieve plate is helical upwards to guide a part of two/three-phase flow in the riser section, and the body of the helical sieve plate is provided with a plurality of sieve meshes to guide the remaining two/three-phase go through the helical sieve plate in the riser section and to break bubbles. The present invention gives consideration to both macroscopic mixing and microscopic mixing processes. In addition to driving liquid to circularly flow by using ejected gas, the helical sieve plate can be used for breaking large bubbles into small bubbles thereby effectively preventing the bubbles from coalescing, increasing gas holdup and increasing a volumetric oxygen transfer coefficient.

### CROSS-REFERENCES AND RELATED APPLICATIONS

This application claims the benefit of priority to Chinese Application No. 201610956295.9, entitled “Airlift Reactor Assembly with Helical Sieve Plate”, filed Oct. 28, 2016, which is herein incorporated by reference in its entirety.

### BACKGROUND OF THE INVENTION

### Field of the Invention

The present invention relates to the fields of bioengineering and environmental engineering, and in particular to an airlift reactor assembly with helical sieve plate.

### Description of the Related Art

Gas-liquid dispersion and mixing are widely used in aerobic fermentation, biological aeration, photoreaction of plant cells and algae cells, and other process units. At present, the reactors capable of implementing the gas-liquid mixing and dispersion mainly include (1) bubble column reactor; (2) airlift reactor; (3) stirred tank reactor; and (4) mixing system based on rotary nozzles.

Since the 1970s, many engineers began to study the bubble column reactor. The bubble column is a column reactor in which a gas sparger is installed at the bottom of the reactor, compressed air is sparged from air holes, and the gas is dispersed in the liquid for mass transfer and heat transfer. It is widely used in the industrial processes such as hydrogenation, desulfurization, waste gas and waste water treatment and microbial culture. It has its advantages such as simple structure, absence of mechanical transmission components, easy sealing and cleaning, stable operation and low energy consumption. However, the bubble column reactor also has some shortcomings, for example, the maximum gas-liquid mass transfer rate in the bubble column is not high, the operating parameter adjustable range is narrow and bubble coalescence is easy to occur, so the mixing efficiency is relative low.

On the basis of the bubble column, many engineers introduced a draft tube into the bubble column to achieve regular circulation of liquid flow, that is, an airlift reactor. In the airlift reactor, high-speed gas is sparged from air nozzle. The gas is dispersed in the liquid in the form of bubbles. On the aeration side, the average liquid density decreases, while the liquid density is retained on the non-aeration side, thus resulting in fluid density difference between the both sides, thereby forming a circulation flow in the reactor. Such a reactor can strengthen the macroscopic mixing to improve the mass transfer efficiency, the agitation of the airflow is stronger than that in the bubble column, and the mixing is more remarkable.

The airlift reactor has the advantages of simple structure, good mass transfer and heat transfer efficiency, low energy consumption, mild shear stress and alleviated damage to cells, and is increasingly used in the bio-chemical reaction process, especially in the aerobic biological reaction process. Statistical estimation of industrial application showed that aerobic fermentation implemented by the airlift bioreactor would save power input by 30 to 50% compared with that of the traditional mechanical stirring bioreactor with the same size. However, the mechanical stirred bioreactor is still widely used in the fermentation industry at present. As bubbles are easy to coalesce in the riser section in the airlift reactor in commercial application and the adjustment range of operating parameters is narrow, the operation flexibility of the reactor is limited. Optimization of airlift reactor structure and improvement of the reaction rate have become a very realistic and urgent need.

Some engineers and researchers assembled stainless steel horizontal wire meshes or sieve plates in the riser section of the airlift reactor to promote bubble breakup and increase the gas holdup. In comparison with the conditions without the assembly component, the volumetric oxygen transfer coefficient increased to about twice that of the control group. However, the installation of horizontal wire mesh or sieve plate in the riser section will reduce the effective operation range of the airlift reactor. When the air flow rate is too high, it is easy to form air blocking beneath the wire mesh or the horizontal sieve plate and is adverse to efficient gas-liquid mass transfer. Furthermore, when the horizontal wire mesh or the sieve plate is installed, it is not easy to clean the inside of the reactor, thus affecting the practical industrial application of the airlift reactor in biological pharmaceutical and food industries.

Aizawa and Takemura invented a gas-liquid contact device with a helical passage (JPH06312126), comprising a helical plate component forming a helical passage. In the device, circular holes with free area ratio of 0.1% to 30% were arranged on the helical plate component, and separation baffles were vertically mounted under the helical plate component in the riser section. The purpose of their invention was to use the helical plate component to guide the gas-liquid fluid in a plug flow pattern therein so as to increase the path length of the gas-liquid fluid between the bottom and the top of the device, thereby extending the residence time of the fluid and allowing the gas-liquid to fully contact in the helical passage. This method by prolonging the gas-liquid contact time has a good performance on the gas absorption of low flow rate gas which was easy to dissolve in a solvent, but was reluctant for the dispersion of high flow rate gas-liquid and insoluble gas such as oxygen and hydrogen.

### SUMMARY OF THE INVENTION

In order to solve the above-mentioned technical problems, the purpose of the present invention is to provide an airlift reactor assembly with helical sieve plate. According to the present invention, macroscopic mixing and microscopic mixing processes are combined, injected gas is utilized to drive liquid to vertical flow, and moreover, the helical sieve plate can be used for breaking the rising large bubbles into small bubbles thereby effectively preventing the bubbles from coalescence, increasing gas holdup and increasing volumetric oxygen transfer coefficient.

In order to achieve the above-mentioned purpose, the present invention provides the following technical solution: an airlift reactor assembly with a helical sieve plate, comprising a reaction tank, the reaction tank being provided with a draft tube and a gas sparger therein, the inner space and the outer space of the draft tube respectively forming a cylindrical guide passage and an annulus-shaped guide passage. One of the guide passages is arranged to be a riser section and the other one is arranged to be a downcomer section, and the gas sparger being arranged just below the riser section, wherein a helical sieve plate is mounted in the riser section. A body of the helical sieve plate is helically upwards to guide a part of two/three-phase flow in the riser section, and the body of the helical sieve plate is provided with a lot of sieve meshes to guide the remaining two/three-phase flow in the riser section and to break bubbles.

In one embodiment, the downcomer section of the draft tube is further provided with a plurality of baffles, and the plurality of baffles is arranged at an inlet of the downcomer section to prevent or weaken a vortex formed during gas-liquid separation.

In one embodiment, the plurality of baffles is evenly arranged in a circumferential direction, and the number of the baffles is between 2 and 8.

In one embodiment, the ratio of the width of the baffle to the diameter of the draft tube is between (0.05 to 1) and (0.15 to 1), and the ratio of the height of the baffle to the diameter of the draft tube is between (0.1 to 1) and (0.5 to 1).

In one embodiment, the side wall of the draft tube is arranged with a plurality of side holes, and the plurality of side holes is evenly distributed within a short flow region of middle and lower portions on the draft tube to allow a small amount of small bubbles in the riser section to enter the downcomer section.

In one embodiment, the holes in short flow region is horizontally annular or helical distributed.

In one embodiment, the width of the short flow region is 50 to 300 mm, the pore diameter of the side hole is 3 to 10 mm, and the free area ratio of the side hole opposed to the short flow region is 20 to 50%, and the ratio of the short flow region area to the cross section area of the draft tube is (0.2 to 1) to (1 to 1).

In one embodiment, the ratio of the pitch of the helical sieve plate to the diameter of the outer trajectory of the sieve plate helical surface is between 1 and 4.

In one embodiment, the ratio of the plate pitch of the helical sieve plate to the diameter of the outer trajectory of the sieve plate helical surface is between 0.5 and 2.

In one embodiment, the pitch of the helical sieve plate is integral multiple of the plate spacing between the adjacent helical sieve plates.

In one embodiment, the ratio of a distance between the lower edge of the helical sieve plate and the bottom of the draft tube to the inner diameter of the reaction tank is between 0.5 and 2.

In one embodiment, the ratio of a distance between the upper edge line of the helical sieve plate and the top of the draft tube to the inner diameter of the reaction tank is between 0.1 and 0.5.

In one embodiment, the ratio of a distance between the bottom of the draft tube and the upper edge of a bottom head of the reaction tank to the inner diameter of the reaction tank is between 0 and 0.3.

In one embodiment, the surface of the helical sieve plate is a regular helical surface, and the projection of the outer trajectory thereof on the draft tube overlaps with the projection of the inner trajectory thereof on the draft tube in the same radial direction.

In one embodiment, the helical surface of the helical sieve plate is an inwardly beveled helical surface, and the projection of the outer trajectory thereof on the draft tube is higher than the projection of the inner trajectory thereof on the draft tube in the same radial direction.

In one embodiment, the helical surface of the helical sieve plate is an outwardly beveled helical surface, and the projection of the outer trajectory thereof on the draft tube is lower than the projection of the inner trajectory thereof on the draft tube in the same radial direction.

In one embodiment, the outer trajectory of a helical surface of the helical sieve plate is an equal-pitch helical line or a variable-pitch helical line.

In one embodiment, the helical sieve plate is helical left-handed or right-handed.

In one embodiment, the free area ratio co of the helical sieve plate is within the range of 20% to 70%.

In one embodiment, the free area ratio co of the helical sieve plate is within the range of 35% to 70%. For the oxygen mass transfer of air in an aqueous solution, the free area ratio is between 60% and 70% preferably, for example 63%.

In one embodiment, the sieve mesh is a square mesh or a polygonal mesh or a circular mesh or an irregularly-shaped mesh.

In one embodiment, the diameter of the sieve mesh is between 2 to 50 mm.

In one embodiment, a ring pipe is adopted as the gas sparger, the ring pipe is arranged with a plurality of air holes evenly distributed on the upper part thereof in the circular direction and the air hole directly faces the riser section; or

In one embodiment, the gas sparger is arranged with a plurality of nozzles distributed in a circumferential direction, the nozzle is a single-port nozzle or a multi-port nozzle, and the nozzle is a gas nozzle or a gas-liquid mixing nozzle. The nozzle directly faces the riser section or the nozzle is a rotary-cut nozzle bending obliquely downward for 45°.

In one embodiment, the helical sieve plate is of a monolithic structure suitable for a small airlift reactor.

In one embodiment, the helical sieve plate is of an assembled structure suitable for a medium-scale or large-scale airlift reactor, and the assembled structure is formed by splicing a plurality of preformed helical sieve plates and is fixed by means of welding or riveting.

In one embodiment, the height-diameter ratio of the internal space of the reaction tank is between (2 to 1) and (6 to 1).

In one embodiment, the reaction tank comprises a two/three-phase mixing zone located on the lower side of the tank and a two/three-phase separation zone located on the upper side of the tank. The inner diameter of the separation zone is not less than that of the mixing zone.

In one embodiment, the cross section area ratio of the riser to the downcomer is between (1 to 0.4) and (1 to 1).

In one embodiment, the airlift reactor is used for gas-liquid reaction.

In one embodiment, the airlift reactor is used for gas-liquid-solid three-phase reaction.

In one embodiment, the air airlift reactor is used for aerobic cultivation of microorganisms, animal cells and plant cells; the ratio of the air flow rate of the reactor to the volume of a culture during aerobic cultivation of microorganisms, animal cells and plant cells is between 0.1 and 3 vvm. When the reactor is a small airlift reactor, such a ratio is biased to the upper limit, and is biased to the lower limit when the reactor is a medium-scale or large-scale airlift reactor. However, the practical operating parameters should be determined based on actual oxygen uptake rate requirements of the microorganisms.

In one embodiment, the top of the reaction tank is provided with a feed inlet, an air outlet, a sight glass, a lamp hole, a spare feed inlet, a manhole, a safety valve and other auxiliaries, and the bottom thereof is provided with a feed outlet.

In one embodiment, the airlift reactor is provided with electrodes such as temperature, pressure, pH, dissolved oxygen, etc. according to the reaction conditions.

In one embodiment, the airlift reactor can be provided with a heat exchange accessories at a suitable location to control the temperature during the reaction, and the heat exchange accessories can be a conventional jacket, a half-pipe coil jacket, a dimple jacket or a plate coil jacket. The heat exchange device can be installed on the outer wall of the reactor or installed on the draft tube or installed on both the outer wall of the reactor and the draft tube.

In one embodiment, when the airlift reactor is applied to a photoreaction process such as cultivation of plant cells and algal cells, the reaction tank, the draft tube and the helical sieve plate are made of transparent materials to be suitable for photoreaction.

In one embodiment, the airlift reactor is a pressure vessel and typically operated under low pressure conditions. The reactor can be operated under medium-pressure conditions when being applied for special chemical reactions. The operating pressure of the reactor is generally 0.2 to 2.0 atm (gauge pressure) when the reactor is used for a biological process.

As a result of the technical solution above, the present invention has the following advantages compared with the prior art.

(1) The helical sieve plate according to the present invention is mounted in the riser section of the airlift reactor to break the rising bubbles and form a helical passage. When bubbles are ejected from the gas sparger, a part of the gas-liquid flow moves upward, and the rising large bubbles are broken by the sieve pores on the sieve plate into small bubbles, thereby significantly reducing the size of the bubbles and improving the gas-liquid mass transfer efficiency. The other part of the gas-liquid flow moves upward along the helical direction of the helical sieve plate. The two parts of fluids interact with each other to form a cross-current flow and a turbulent flow, which enhances the radial and axial micro-mixing, helps to prevent bubble coalescence and reduces the bubble size. Compared with the horizontal sieve plate, the fluid resistance of the rising flow is smaller, the air blocking and slug flow are not easy to occur, and the effective operating range of the reactor is widened. The helical passage enables the gas-liquid fluid to form a circulation, thereby promoting the macroscopic mixing. The installation of the helical sieve plate also improves the gas-liquid mass transfer performance, i.e., significantly improves the gas holdup and volumetric oxygen transfer coefficient.

(2) The appropriate free area ratio of the helical sieve plate prevents bubbles from coalescing into a large bubble and ensures excellent gas-liquid mass transfer efficiency.

(3) The baffle according to the present invention is mounted at the inlet of the downcomer section. After a two/three-phase flow ascends to the two/three-phase separation zone, the flow will form a vortex, and the baffle can prevent or weaken the vortex flow at the inlet of the downcomer section. In addition, for the biological reaction process, especially the aerobic fermentation process, the baffle is arranged in the downcomer section and a smooth structure is maintained in the riser section, so it prevents the small bubbles from coalescing into large bubbles and further forming the slug flow under the sieve plate. It is convenient to thoroughly clean the insider of device and avoids the microbiological contamination due to dead zone for sanitation.

(4) The side hole according to the present invention is arranged at a middle or lower region of the draft tube so that a small amount of small bubbles in the riser section can enter the downcomer section, and the small bubbles are caught by the liquid flow in the downcomer section to flow downwards, thereby improving the gas-liquid mass transfer process of the downcomer section without affecting the circulation flow of the downcomer section so as to improve the overall gas-liquid mass transfer efficiency of the airlift reactor.

(5) The airlift reactor according to the present invention has the characteristics of low energy consumption, high mass transfer efficiency and mild shear stress, and is suitable for the process of biological aeration and aerobic fermentation, especially suitable for the submerged cultivation of mold, actinomycetes, animal cells, algae cells and plant cells that are sensitive to shear stress. In the process of microbial culture, the sieve mesh is not easy to be gradually clogged by the growing bacteria, which is conducive to the cultivation of microorganisms.

### BRIEF DESCRIPTION OF DRAWINGS

FIG. 1 is a cross-sectional view of an airlift reactor disclosed in Example 1 of the present invention;

FIG. 2 is an installation diagram of a draft tube and a helical sieve plate disclosed in Example 1 of the present invention;

FIG. 3 is a top view of a gas sparger disclosed in Example 1 of the present invention;

FIG. 4 is a cross-sectional view of an airlift reactor disclosed in Example 2 of the present invention;

FIG. 5 is a decomposition diagram of a draft tube and a helical sieve plate disclosed in Example 2 of the present invention;

FIG. 6 is a top view of a gas sparger disclosed in Example 2 of the present invention;

FIG. 7 is a figure showing the relationship between the free area ratio co and the volumetric oxygen transfer coefficient as disclosed in Example 4 of the present invention.

Where: 10: reaction tank; 11: gas-liquid separation zone; 12: gas-liquid mixing zone; 121: cylindrical downcomer section; 122: annular riser section; 123: cylindrical riser section; 124: annular downcomer section; 13: feed inlet; 14: air outlet; 15: feed outlet; 16: air inlet pipe; 20: draft tube; 21: baffle; 22: short flow region; 30: gas sparger; 31: air hole; 40: helical sieve plate; 41: sieve mesh; 42 inner trajectory; 43: outer trajectory; 44: upper edge line; 45: lower edge line; 46: sieve plate helical surface; 50: jacket; 51: cooling water outlet; and 52: cooling water inlet.

### DETAILED DESCRIPTION

The present invention is further described below with reference to the accompanying drawings and embodiments. The following embodiments are intended to illustrate the present invention, but are not intended to limit the scope of the present invention.

The volumetric oxygen transfer coefficient was measured by dynamic gas out method (IEEE Access, 2017, 5: 2711-2719.). First, nitrogen gas was injected into the reactor to remove the oxygen originally dissolved in the water until the reading of a dissolved oxygen electrode was less than 5%. Afterwards, the nitrogen gas feeding was stopped and then the air was injected at a preset flow rate, a dissolved oxygen controller instrument automatically collected the dissolved oxygen reading every 5s until the dissolved oxygen reading reached 90% or more. The measurement stopped after the dissolved oxygen concentration was saturated. In consideration of the response time of the dissolved oxygen electrode, the volumetric oxygen transfer coefficient was calibrated by the equation as follows,

$$\frac{{C}^{*} - {C}_{L}}{{C}^{*} - {C}_{0}} = \frac{{e}^{- {k}_{L} a \cdot t} - {k}_{L} a \cdot {τ}_{e} \cdot {e}^{- \frac{t}{{τ}_{e}}}}{( 1 - {k}_{L} a \cdot {τ}_{e} )}$$

wherein said C* is saturated dissolved oxygen level, %; C_{L} is the measured dissolved oxygen concentration, %; C₀ is the initial dissolved oxygen concentration, %; t is the measuring time, s; and Te is the response time of the electrode, s.

### EXAMPLE 1

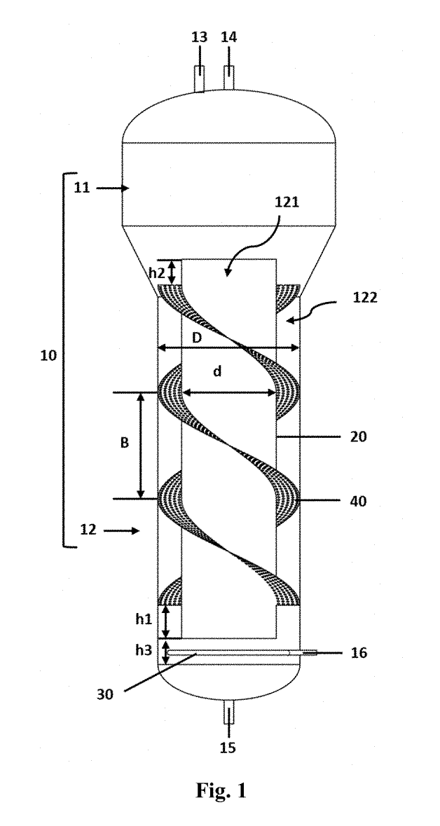

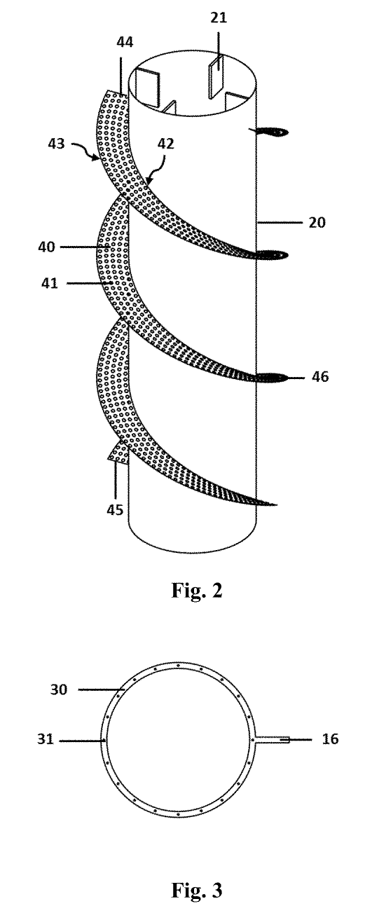

Referring to FIG. 1 to FIG. 3, an airlift reactor with a helical sieve plate is used for gas-liquid reaction as shown in the legends in the figures. The airlift reactor comprises a reaction tank 10, the internal space of the reaction tank 10 is divided into a gas-liquid mixing zone 12 located on the lower side and a gas-liquid separation zone 11 located on the upper side. The gas-liquid mixing zone 12 is provided with a draft tube 20 and a gas sparger 30 on upper and lower portions therein. The draft tube 20 and the reaction tank 10 are coaxially arranged to divide the gas-liquid mixing zone 12 into a cylindrical downcomer section 121 located inside of the draft tube 20 and an annular riser section 122 located outside the draft tube 20, and the gas sparger 30 introduces air into the annular riser section 122. The airlift reactor also comprises a helical sieve plate 40 mounted in the round riser section 122, the body of the helical sieve plate 40 is helical upwards, and sieve meshes 41 are densely distributed on the body of the helical sieve plate 40.

The reaction tank 10 is provided with a feed inlet 13 and an air outlet 14 at the top, and a feed outlet 15 and an air inlet pipe 16 at the bottom.

The ratio of the pitch p of the helical sieve plate 40 to the inner diameter D of the reaction tank 10 is 2.

The ratio of the plate spacing B between the adjacent helical sieve plates 40 to the inner diameter D of the reaction tank 10 is 1.

The ratio of a distance hl between a lower edge line 45 of the helical sieve plate 40 and the bottom of the draft tube 20 to the inner diameter D of the reaction tank 10 is 0.5.

The ratio of a distance h2 between an upper edge line 44 of the helical sieve plate 40 and the top of the draft tube 20 to the inner diameter D of the reaction tank 10 is 0.2.

The ratio of a distance h3 between the bottom of the draft tube 20 and a bottom head of the reaction tank 10 to the inner diameter D of the reaction tank 10 is 0.1 to 1.

As the helical sieve plate 40 has a uniform thickness, the structural features of the helical sieve plate 40 can be represented by a sieve plate helical surface 46. The sieve plate helical surface 46 is defined by an inner trajectory 42, an outer trajectory 43, an upper edge line 44 and a lower edge line 45. The projection of the outer trajectory 43 on the draft tube 20 overlaps with the inner trajectory 42, and the sieve plate helical surface 46 is formed by enabling the lower edge line 45 as the generatrix sliding along the outer trajectory 43. The outer trajectory of the helical surface of the helical sieve plate 40 is an equal-pitch helical line.

The free area ratio of the helical sieve plate 40 is 50%.

A plurality of sieves meshes 41 is evenly distributed, and any three adjacent sieve meshes 41 are located at the three vertexes of a regular triangle.

The sieve mesh 41 is a circular mesh and the diameter of the circular mesh is 5 mm.

The cylindrical downcomer section 121 is provided with a plurality of baffles 21 evenly arranged on an upper end of the draft tube 20 in the circumferential direction.

The baffles 21 are vertically downward.

The number of the baffles 21 is four.

The ratio of the width of the baffle 21 to the diameter of the draft tube 20 is 0.1 to 1.

The ratio of the height of the baffle 21 to the diameter of the draft tube 20 is 0.25 to 1.

A ring pipe is adopted as the gas sparger 30, the ring pipe is arranged with a plurality of air holes 31 which are evenly arranged in the upper portion thereof in the circumferential direction, and the air holes 31 directly face the annular riser section 122.

The height-diameter ratio of the gas-liquid mixing zone 12 component in the reaction tank 10 is 3 to 1.

The ratio of the inner diameter of the gas-liquid separation zone 11 to the inner diameter of the gas-liquid mixing zone 12 is 1.2 to 1.

The ratio of the cross section area of the annular riser section 122 to the cylindrical downcomer section 121 is 1 to 0.8, that is, the ratio of the inner diameter d of the draft tube 20 to the inner diameter D of the reaction tank 10 is 2 to 3.

When the present embodiment was used, the volumetric oxygen transfer coefficients measured under different superficial gas velocity conditions were shown in Table 1:

TABLE 1
Volumetric oxygen transfer coefficients under
different superficial gas velocity conditions

| | | | | | |
|---|---|---|---|---|---|
| Superficial gas velocity | 0.009 | 0.027 | 0.045 | 0.063 | 0.081 |
| (m/s) | | | | | |
| Volumetric oxygen transfer | 0.0086 | 0.0328 | 0.0603 | 0.0857 | 0.121 |
| coefficient (s⁻¹) | | | | | |

The airlift reactor is a pressure vessel, and is generally operated under low pressure conditions when it is used for biological aerobic fermentation and cultivation of plant cells. The reactor can be operated under medium-pressure conditions when being applied for specific chemical reaction.

When air is introduced from the air inlet pipe 16, bubbles are ejected from the air hole 31, after which, a part of the gas-liquid flow rises upward, and the bubbles meeting the sieve plate will be broken into small bubbles. The other part of the gas-liquid flow helically rises along the sieve plate. The two parts interact with each other to form a cross-current flow and turbulent flow, which helps to prevent the bubbles from coalescing and reduce the size of the bubble. When the liquid reaches the top, it will helically flow to reduce the liquid velocity. A baffle 21 is additionally arranged in the draft tube 20, which can weaken the vortex flow of liquid in the downcomer section.

### EXAMPLE 2

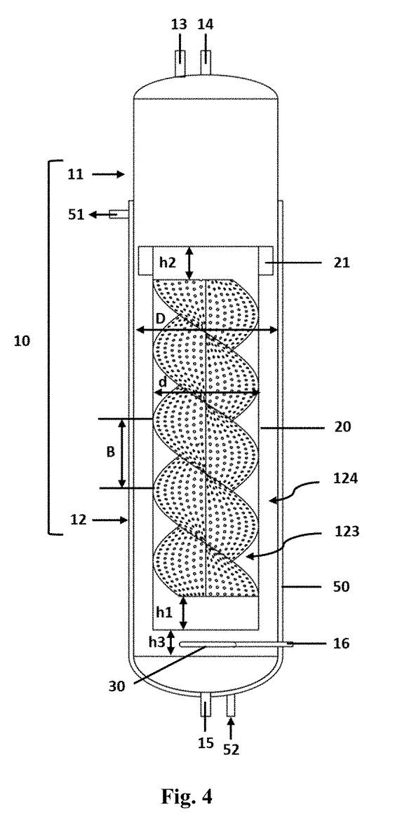

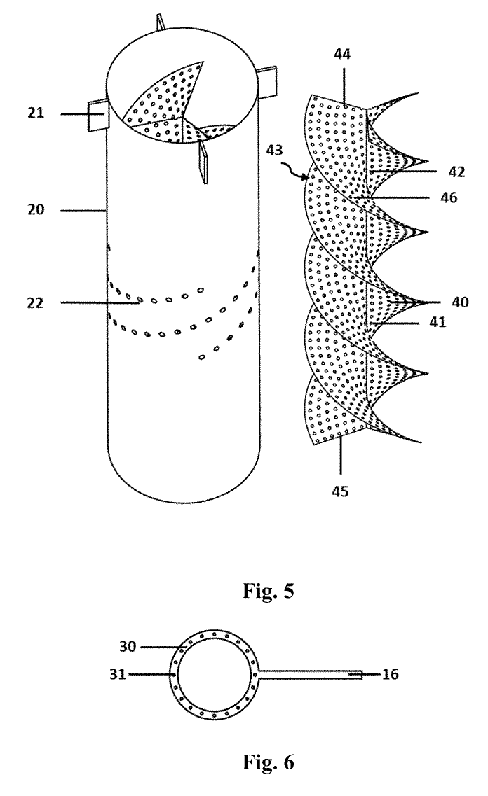

Referring to FIG. 4 to FIG. 6, an airlift reactor with a helical sieve plate is used for gas-liquid reaction as shown in the legends in the figures. The airlift reactor comprises a reaction tank 10, and the internal space of the reaction tank 10 is divided into a gas-liquid mixing zone 12 located on the lower side and a gas-liquid separation zone 11 located on the upper side. The gas-liquid mixing zone 12 is provided with a draft tube 20 in the upper side and a gas sparger 30 in the lower side therein. The draft tube 20 and the reaction tank 10 are coaxially arranged to divide the gas-liquid mixing zone 12 into a cylindrical downcomer section 123 located inside of the draft tube 20 and an annular riser section 124 located outside the draft tube 20, and the gas sparger 30 introduces air into the annular riser section 123. The airlift reactor also comprises a helical sieve plate 40 mounted in the cylindrical riser section 123, the body of the helical sieve plate 40 is helical upwards, and sieve meshes 41 are distributed on the body of the helical sieve plate 40.

The reaction tank 10 is provided with a feed inlet 13 and an air outlet 14 at the top, and a feed outlet 15 and an air inlet pipe 16 at the bottom.

The ratio of the pitch p of the helical sieve plate 40 to the diameter d of the draft tube 20 is 1.8.

The ratio of the plate spacing B between the adjacent helical sieve plates 40 to the diameter d of the draft tube 20 is 0.6.

The ratio of a distance h1 between a lower edge line 45 of the helical sieve plate 40 and the bottom of the draft tube 20 to the inner diameter D of the reaction tank 10 is 0.5.

The ratio of a distance h2 between an upper edge line 44 of the helical sieve plate 40 and the top of the draft tube 20 to the inner diameter D of the reaction tank 10 is 0.2.

The ratio of a distance h3 between the bottom of the draft tube 20 and a bottom head of the reaction tank 10 to the inner diameter D of the reaction tank 10 is 0.1 to 1.

As the helical sieve plate 40 has a uniform thickness, the structural features of the helical sieve plate 40 can be represented by a sieve plate helical surface 46. The sieve plate helical surface 46 is defined by an inner trajectory 42, an outer trajectory 43, an upper edge line 44 and a lower edge line 45. The projection of the outer trajectory 43 on the draft tube 20 overlaps with the inner trajectory 42, and the sieve plate helical surface 46 is formed by enabling the lower edge line 45 as the generatrix sliding along the outer trajectory 43. The outer trajectory of the helical surface of the helical sieve plate 40 is an equal-pitch helical line.

The free area ratio of the helical sieve plate 40 is 50%.

A plurality of sieves meshes 41 is evenly distributed, and any three adjacent sieve meshes 41 are located at the three vertexes of a regular triangle.

The sieve mesh 41 is a circular mesh and the diameter of the circular mesh is 5 mm.

The cylindrical downcomer section 121 is provided with a plurality of baffles 21 evenly arranged on an upper end of the draft tube 20 in the circumferential direction.

The baffles 21 are vertically downward.

The number of the baffles 21 is four.

The ratio of the width of the baffle 21 to the diameter of the draft tube 20 is 0.1 to 1.

The ratio of the height of the baffle 21 to the diameter of the draft tube 20 is 0.25 to 1.

A short flow region 22 can be arranged at the middle and lower portions of the draft tube 20, and the short flow region 22 is helical and is provided with a plurality of side holes. The height of the short flow region 22 is 150 mm, the diameter of the small hole in the short flow region 22 is 8 mm, and the free area ratio is 20%. The ratio of the area of the short flow region 22 to the cross section area of the draft tube is 0.2 to 1.

A ring pipe is adopted as the gas sparger 30, the ring pipe is provided with a plurality of air holes 31 which are evenly arranged in the upper portion thereof in the circumferential direction, and the air holes 31 directly face the cylindrical riser section 123.

The height-diameter ratio of the internal space of the reaction tank 10 is 4 to 1.

The inner diameter of the gas-liquid separation zone 11 is greater than the inner diameter of the gas-liquid mixing zone 12.

The ratio of the cross section area of the annular riser section 123 to the cross section area of the cylindrical downcomer section 124 is 1 to 0.8, that is, the ratio of the inner diameter d of the draft tube 20 to the inner diameter D of the reaction tank 10 is 2 to 3.

A jacket 50 is mounted outside the reaction tank 10, and a cooling water inlet 52 and a cooling water outlet 51 are respectively located therebelow and thereabove.

The airlift reactor is a pressure vessel and typically operated under low pressure conditions, and the reactor can be operated under medium-pressure conditions when being used for specific chemical reaction.

When air is introduced from the air inlet pipe 16, bubbles are ejected from the air hole 31, after which, a part of the gas-liquid flow rises upward, and the bubbles meeting the sieve plate will be broken into small bubbles. The other part of the gas-liquid flow helically rises along the sieve plate. The two parts interact with each other to form a cross-current flow and turbulent flow, which helps to prevent the bubbles from coalescing and reduce the size of the bubble. When the liquid reaches the top, it will helically flow to reduce the liquid velocity. Baffles 21 are additionally arranged outside the draft tube 20, which can prevent the liquid from helically flowing.

### EXAMPLE 3

The remaining is the same as the embodiment 1. The difference is that the airlift reactor in the present embodiment is used for aerobic cultivation of microorganisms, animal cells and plant cells. The ratio of air flow (m³/min) to culture solution volume (m³) is 0.1 to 3. When the reactor is a small reactor, the ratio is biased to the upper limit. During medium-scale and large-scale reaction, the ratio is biased to the lower limit, but the specific operating parameters should be determined based on actual oxygen uptake rate requirements of the microorganisms. The operating pressure (gauge pressure) is generally from 0.2 to 2.0 atm.

In one embodiment, the reaction tank, the draft tube and the helical sieve plate are made of transparent materials to be suitable for photoreaction.

### EXAMPLE 4

An airlift reactor with a helical sieve plate is provided. As shown in FIG. 1, the inner cylinder diameter D of the reaction tank is 370 mm, and the cylinder height of the reaction tank is 1130 mm. The upper and lower head are standard elliptical heads. The draft tube in the reaction tank has a height of 750 mm, an inner diameter d of 230 mm and a wall thickness of 5 mm. The distance h3 between the lower edge of the draft tube and the lower edge of the cylinder of the reaction tank is 100 mm, the distance between the upper edge of the draft tube and the liquid level is 50 mm, the distance h2 between upper edge of the helical sieve plate and the upper edge of the draft tube is 90 mm, the distance hl between lower edge of the helical sieve plate and the lower edge of the draft tube is 60 mm, the pitch p of the helical sieve plate is 600 mm, and the plate spacing B between the adjacent helical sieve plates is 200 mm.

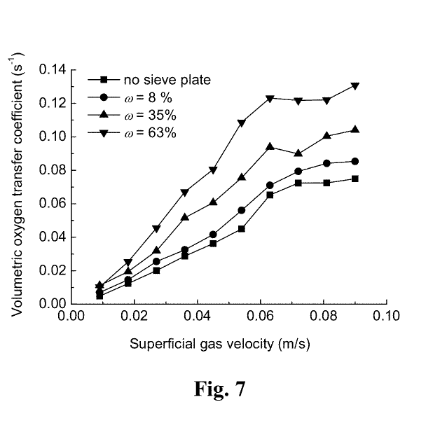

FIG. 7 shows volumetric oxygen transfer coefficients of an airlift reactor without a helical sieve plate and an airlift reactor provided with different helical sieve plates with free area ratios of 8%, 35% and 63% respectively. It can be seen that the assembly of the helical sieve plate can significantly improve the oxygen transfer performance. In the range of the free area ratio investigated, improving the free area ratio can intensify the mass transfer performance. When the free area ratio of the helical sieve plate is 63% and the superficial gas velocity is 0.09 m/s, the volumetric oxygen transfer coefficient of the airlift reactor reaches 0.131 s⁻¹. It increases by 45 to 80% compared with that of the free area ratio is 8%, and increases by 68 to 110% compared with that of without helical sieve plate.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

1. An airlift reactor assembly with a helical sieve plate, comprising a reaction tank, wherein the reaction tank being assembled with a draft tube and a gas sparger therein; wherein an inner space and an outer space of the draft tube respectively forming a cylindrical guide passage and an annulus-shaped guide passage; wherein one of the cylindrical guide passage and the annulus-shaped guide passage being arranged to be an riser section and the other one being arranged to be a downcomer section, and the gas sparger being arranged just below the riser section; wherein a helical sieve plate is mounted in the riser section of the draft tube; wherein a body of the helical sieve plate is helical upwards to guide a part of two/three-phase flow in the riser section, and the body of the helical sieve plate is provided with a plurality of sieve meshes to guide the remaining two/three-phase flow go through the helical sieve plate in the riser section and to break bubbles.

2. The airlift reactor according to claim 1, wherein the downcomer section is further provided with a plurality of baffles, and the plurality of baffles are arranged at an inlet of the downcomer section to prevent or weaken a vortex formed during gas-liquid separation.

3. The airlift reactor according to claim 2, wherein the plurality of baffles are evenly arranged in circumferential direction, and the number of the baffles is between 2 and 8.

4. The airlift reactor according to claim 2, wherein a ratio of a width of the baffle to a diameter of the draft tube is between (0.05 to 1) and (0.15 to 1), and a ratio of a height of the baffle to a diameter of the draft tube is between (0.1 to 1) and (0.5 to 1).

5. The airlift reactor according to claim 1, wherein a side wall of the draft tube is arranged with a plurality of side holes, and the plurality of side holes are evenly distributed within a short flow region on middle and lower portions of the draft tube to allow a small amount of small bubbles in the riser section to enter the downcomer section directly.

6. The airlift reactor according to claim 5, wherein the short flow region is an annular or helical band on the draft tube.

7. The airlift reactor according to claim 5, wherein a width of the short flow region is 50 to 300 mm, a diameter of the side hole is 3 to 10 mm, a free area ratio of the short flow region is 20 to 50%, and a area ratio of the short flow region area to the cross section area of the draft tube is (0.2 to 1) to (1 to 1).

8. The airlift reactor according to claims 1, wherein a ratio of the pitch of the helical sieve plate to a diameter of an outer trajectory of the sieve plate is between 1 and 4, or a ratio of the plate pitch of the helical sieve plate to a diameter of an outer trajectory of the sieve plate is between 0.5 and 2, or a pitch of the sieve plate is integral multiple of plate spacing between adjacent helical sieve plates.

9. The airlift reactor according to claims 1, wherein a ratio of a distance between the lower edge line of the helical sieve plate and a bottonn of the draft tube to an inner diameter of the reaction tank is between 0.5 and 2, a ratio of the distance between an upper edge line of the helical sieve plate and a top of the draft tube to an inner diameter of the reaction tank is between 0.1 and 0.5, and a ratio of a distance between a bottom of the draft tube and an upper edge of the bottom head of the reaction tank to an inner diameter of the reaction tank is between 0 and 0.3.

10. The airlift reactor according to claims 1, wherein a surface of the helical sieve plate is a regular helix surface, and a projection of an outer trajectory thereof on the draft tube coincides with a projection of an inner trajectory thereof on the draft tube in a same radial direction; or a helix surface of the helical sieve plate is an inwardly beveled helix surface, and a projection of an outer trajectory thereof on the draft tube is higher than a projection of an inner trajectory thereof on the draft tube in a same radial direction; or a helix surface of the helical sieve plate is an outwardly beveled helix surface, and a projection of an outer trajectory thereof on the draft tube is lower than a projection of an inner trajectory thereof on the draft tube in a same radial direction.

11. The airlift reactor according to claims 1, wherein an outer trajectory of a helix surface of the helical sieve plate is an equal-pitch helix line or a variable-pitch helix line, and the helical sieve plate comprises left-handed or right-handed helix surface.

12. The airlift reactor according to claims 1, wherein a free area ratio of the helical sieve plate is within a range of 20% to 70%; wherein a sieve mesh is a square mesh or a polygonal mesh or a circular mesh or a irregularly-shaped mesh and a diameter of the sieve mesh is between 2 to 50 mm.

13. The airlift reactor according to claims 1, wherein a free area ratio of the helical sieve plate is within a range of 35% to 70%; wherein a sieve mesh is a polygonal mesh or a circular mesh or an irregularly-shaped mesh and a diameter of the sieve mesh is between 2 to 50 mm.

14. The airlift reactor according to claims 1, wherein a free area ratio of the helical sieve plate is 63%; wherein a sieve mesh is a polygonal mesh or a circular mesh or an irregularly-shaped mesh and a diameter of the sieve mesh is between 5 to 40 mm.

15. The airlift reactor according to claims 1, wherein a ring pipe is adopted as the gas sparger; wherein the ring pipe is provided with a plurality of air holes evenly distributed on an upper part thereof in a circular direction and the air holes directly face the riser section.

16. The airlift reactor according to claim 15, wherein the gas sparger is provided with a plurality of nozzles arranged in a circumferential direction; wherein a nozzle of the plurality of nozzles is a single-port nozzle or a multi-port nozzle; wherein a nozzle of the plurality of nozzles is a gas nozzle or a gas-liquid mixing nozzle, and a nozzle of the plurality of nozzles directly faces the riser section or a nozzle of the plurality of nozzles is a rotary-cut nozzle bending obliquely downward for 45°.

17. The airlift reactor according to claims 1, wherein the helical sieve plate is of a monolithic structure suitable for a small airlift reactor or of an assembled structure suitable for a medium-scale or large-scale airlift reactor, and the assembled structure is formed by splicing a plurality of preformed helical sieve plates and is fixed by means of welding or riveting.

18. The airlift reactor according to claims 1, wherein a height-diameter ratio of an internal space of the reaction tank is between 2˜6, and the space comprises a two/three-phase mixing zone located on a lower side and a two/three-phase separation zone located on an upper side; wherein an inner diameter of the reaction tank corresponding to the two/three-phase separation zone is not less than that of the reaction tank corresponding to the two/three-phase mixing zone, and a ratio of a cross section area of the riser section to a cross section area of the downcomer section is between (1 to 0.4) and (1 to 1).

19. The airlift reactor according to claims 1, wherein the air airlift reactor is used for biological aeration and aerobic cultivation of microorganisms, animal cells and plant cells; and wherein the ratio of the air flow rate to the liquid volume during cultivation of microorganisms, animal cells and plant cells is between 0.1 and 3 vvm.
